(19) United States
(12) Patent Application Publication
(10) Pub. No.: US 2013/0323190 A1
(43) Pub. Date: Dec. 5, 2013

(54) COMPOSITION GENERATING HYDROGEN

(71) Applicants: Nobuko Ohta, Kawasaki-shi (JP); Fumiaki Ohta, Koganei-shi (JP); Hiroshi Uesugi, Yokohama-shi (JP)

(72) Inventors: Nobuko Ohta, Kawasaki-shi (JP); Fumiaki Ohta, Koganei-shi (JP); Hiroshi Uesugi, Yokohama-shi (JP)

(73) Assignees: HYDROGEN HEALTH MEDICAL LABO CO., LTD., Tokyo (JP); BIO COKE LAB. CO. LTD., Tokyo (JP)

(21) Appl. No.: 13/985,879

(22) PCT Filed: Feb. 17, 2012
(86) PCT No.: PCT/JP2012/053891

§ 371 Date: Aug. 19, 2013

(30) Foreign Application Priority Data
Feb. 17, 2011 (JP) 2011-032633
Jun. 6, 2011 (JP) 2011-126690

(51) Int. Cl. A61K 8/19; A61K 33/06; A61Q 19/02

(52) U.S. Cl. CPC A61K 8/19; A61Q 19/02; A61K 33/06
USPC 424/62; 424/682

(57) ABSTRACT

There is provided an external-use composition that is incorporated with a hydrogen storing metal alloy keeping the hydrogen and generating the hydrogen, and that has the antioxidant effect, anti-inflammation effect and antiallergic effect.

There is provided a skin external-use composition that is incorporated with a fine particle of hydrogen storing alloyed metal, such as magnesium hydride.

This application is the national phase under 35 U.S.C. §371 of PCT International Application No. PCT/JP2011/053891which has an International filing date of Jun. 16, 2011 and designated the United States of America.

### FIELD

The present invention relates to a skin external-use composition that is incorporated with a metal or metal alloy storing hydrogen showing the antioxidant effect, anti-inflammatory effect and antiallergic effect and that generates hydrogen.

### BACKGROUND

It is known that a molecular hydrogen (represented by H₂ the chemical formula) has the antioxidant effect. Thus, the molecular hydrogen is expected to have advantage for the health enhancement and the therapeutic and preventive effects to several diseases. Furthermore, it is known that the molecular hydrogen has the anti-inflammatory effect and the antiallergic effect. Moreover, it is shown that the molecular hydrogen has the age registering effect, such as to reduce skin wrinkles.

Water, drinking water, and cosmetics in which the molecular hydrogen is solved are available in the market and are widely utilized. However, it is inconvenient to carry the hydrogen gas cylinder or liquid solution in which the molecular hydrogen is dissolved. In addition, the safety violation of burning or exploding may cause when much of hydrogen gas is utilized or much of molecular hydrogen is generated. Even if the actual density of hydrogen is too low to cause such a safety violation, the utilization of hydrogen gas cylinder or liquid solution may be rejected in view of the security.

In the case of skin lotion including molecular hydrogen and applied to skin, the hydrogen gas is evaporated. Thus, much of the molecular hydrogen goes out of the skin, i.e., goes to the atmosphere, without going into the skin. Therefore, it is difficult to keep the molecular hydrogen for a long time, and thus the significant effect can't be not expected.

The blackness of skin due to the sunspot, freckle, scar and the like is caused by the melanin, and thus the inhibitor of melanin synthesis is utilized for the whitening cosmetics. However, the strong reducing agent is required for directly reducing the melanin to perform the lightning. Thus, it is difficult to directly perform the lightning of melanin. Because of difficulty in directly performing the lightning of melanin, it is difficult to make the skin become white in a short time (see Japanese Patent Application Laid-Open No. 2006-151943).

The metal generally keeps atomic hydrogen (single atom represented by H) on the surface or at the inside. In the metals, the palladium and magnesium can keep much atomic hydrogen at the inside, and the nickel and platinum can keep atomic hydrogen on the surface. The atomic hydrogen has the strong reduction effect. Thus, a nickel sponge having a large surface area is industrially utilized for the hydrogenation reaction (see Japanese Patent Application Laid-Open No.H9-132536).

When alloyed metal storing hydrogen is utilized for the cosmetics, it is necessary to liberate the hydrogen (meaning not only the molecular hydrogen but also the atomic hydrogen) from the metal. The palladium, nickel and platinum are not preferable in view of the safety, because a metal insoluble to water remains in the body after the hydrogen is generated from the palladium, nickel and platinum (see Japanese Patent Application Laid-Open No. 2006-151943).

When reacting with water, the magnesium hydride (generally represented by MgH₂ the chemical formula, and having the non-constant H content 0 to 2) can generate the molecular hydrogen based on the reaction formula described below. However, it is required for the generation of molecular hydrogen to keep the high temperature not less than 80 C.° or to apply a catalyst, such as MgCl₂ or acid. The prior patent application applies acid for generating hydrogen from MgH₂.

MgH₂+2H₂O→Mg(OH)₂+2H₂

As described above, the atomic hydrogen has the strong reduction power. However, it is difficult to take the metal alloy storing hydrogen in a cell with keeping the hydrogen in the alloyed metal. Thus, only the molecular hydrogen is utilized for the cosmetics. Even if the cell can take in such an alloyed metal with nanoparticulate form, a metal may remain in the body. Such a remaining metal may be harmful to the organism.

It is not reported about cosmetics, quasi-drug and medical product that generate hydrogen and lead the hydrogen's effect by applying the molecular hydrogen and atomic hydrogen directly onto the skin.

### SUMMARY

The present invention has an object to provide an external-use composition that is incorporated with a hydrogen storing metal or metal alloy which keeps hydrogen therein and which can generate the kept hydrogen.

The inventors in this application has performed the keen examination for a method directly applying the hydrogen onto the skin. The inventors have focused on the utilization of metal or metal alloy storing the hydrogen, and has found that the magnesium hydride (MgH₂) can be utilized for applying the hydrogen onto the skin.

MgH₂ does not react with the oxygen in the air during a short time, which is different from Mg the metal. MgH₂ is micronized by repeated collision among particles thereto. Based on the collision speed and collision frequency, it is possible to produce a particle having a predetermined size. It is further possible to produce a nanoparticle that can pass the superficial skin.

In addition, the specific gravity of MgH₂ is about 1.7. Thus, when MgH₂ is suspended in the glycerol whose specific gravity is 1.26, the particles is precipitated and thus it is not possible to keep the homogenous condition. The suspension including a precipitated material is not preferable as the trade article, because the user needs stirring the suspension when using it. The inventors in this application has performed the keen examination for manufacturing the homogenous suspension. Then, the inventors have found that MgH₂ having the particle diameter not more than 2 μm can implement the high specific gravity, and further prevent the generation of precipitate during a long time in the suspension, such as glycerol or Bayu (horse oil).

The inventors have further found that, when the metal alloy storing hydrogen is suspended and directly applied with homogenous smooth liquid solution or creamy material onto the skin, the hydrogen is absorbed into the skin. Furthermore, the inventors have found that the hydrogen is generated by the reaction with water included in the skin or generated from the skin cell, and then the generated hydrogen is absorbed in the body before dispersed into the air. Moreover, the inventors have found that the magnesium hydride becomes Mg(OH)₂ after reacting with water, and that the Mg(OH)₂ is easily absorbed and flowed out because it is soluble in water although the solubility is poor. Thus, the inventors have additionally found that the utilization of magnesium hydride is safety because it causes no disorder on the skin, and then the inventors have made the present invention. In other words, the present invention is described below:

[1] the skin external-use composition incorporated with a fine particle of metal or metal alloy storing hydrogen;
[2] the skin external-use composition according to [1], wherein the fine particle of metal or metal alloy storing hydrogen is a fine particle of magnesium hydride storing atomic hydrogen;
[3] the skin external-use composition according to [2], which is incorporated with a fine particle of magnesium hydride that can generate molecular hydrogen without catalyst when applied to a skin;
[4] the skin external-use composition according to [2], which is incorporated with a fine particle of magnesium hydride storing atomic hydrogen that passes a superficial skin;
[5] the skin external-use composition according to any one of [1]-[4], which is in a cream state or gel state;
[6] the skin external-use composition according to any one of [1]-[5], which is utilized for whitening;
[7] the skin external-use composition according to any one of [1]-[5], which is utilized for treating a disorder selected from a group of inflammation, allergy, wound, burn injury and bruise;
[8] the skin external-use composition according to any one of [1]-[7], which is selected from a group of cosmetics, massage supporting liquid, esthetic clinic supporting liquid, quasi-drug and medical product;
[9] a homogenous composition that includes a fine particle of magnesium hydride, wherein the fine particle of magnesium hydride is homogenously suspended in an anhydrous liquid;
[10] The composition homogenously suspended in the anhydrous liquid according to [9] for manufacturing a liquid solution having a high density of dissolved molecular hydrogen;
[11] the composition according to [9] or [10], wherein the anhydrous liquid is glycerol or oil;
[12] a method for manufacturing a water liquid solution including hydrogen, comprising applying the composition according to any one of [9]-[11] into a water liquid solution; and
[13] the method according to [12], wherein the water liquid solution is a drink or cosmetics.

When the metal alloy storing hydrogen of the present invention is applied to the skin, the hydrogen is generated and the generated hydrogen leads the antioxidant effect, anti-inflammatory effect and antiallergic effect. As the magnesium hydride of the present invention can be kept and utilized with a homogenous suspension form for a long time, the user does not need stirring it when using it. Therefore, it is convenient. In addition, the water liquid solution dissolving the hydrogen can be made with a room temperature and a short time about 1 minute, by adding the magnesium hydride into the water liquid solution. It will be utilized in a wider range, depending on user's idea. Furthermore, it can be utilized as the externally applied composition, such as a cream which is homogenous, smooth, malleable and comfortable.

The composition of the present invention generating the hydrogen can inhibit the skin aging with the antioxidant effect. In addition, when it is applied to the skin, it leads the whitening effect. Thus, it can be utilized as the whitening cosmetics. Furthermore, it can inhibit the inflammation and thus has the healing acceleration effect for wound or burn injury.

The above and further objects and features will more fully be apparent from the following detailed description with accompanying drawings.

### BRIEF DESCRIPTION OF THE DRAWINGS

FIG. 1A is a graph that shows a distribution of particle diameter of MgH₂ particle before micronized.

FIG. 1B is a graph that shows a distribution of particle diameter of MgH₂ particle after micronized.

FIG. 2 is a view that shows a melanin lighting effect of a composition incorporated with magnesium hydride.

FIG. 3A is a view that shows a skin whitening effect of the composition incorporated with magnesium hydride.

FIG. 3B is a view that shows a skin whitening effect of the composition incorporated with magnesium hydride.

FIG. 3C is a view that shows a skin whitening effect of the composition incorporated with magnesium hydride.

FIG. 4 is a view that shows an effect for wound of the composition incorporated with magnesium hydride.

FIG. 5 is a view that shows an effect for burn injury of the composition incorporated with magnesium hydride.

FIG. 6 is a view that shows an effect for bruise of the composition incorporated with magnesium hydride.

FIG. 7 is a view that shows a measured result of hydrogen density in the composition incorporated with magnesium hydride.

### DETAILED DESCRIPTION

The present invention is explained below in detail.

A composition of the present invention is a fine particle of metal or metal alloy storing and keeping hydrogen. In the present invention, the hydrogen means atomic hydrogen and/or molecular hydrogen.

The molecular hydrogen is generated from the metal or metal alloy storing the hydrogen, when the suspension including the hydrogen storing metal or metal alloy keeping the hydrogen is directly contacted with the skin. Thus, the generated hydrogen is absorbed through the skin into the organism, without dispersing outside the organism. It is known that the molecular hydrogen is dispersed and taken into the cell through the skin surface. In addition, it is known that a fine particle metal is taken into the dermal cell through the superficial skin. Therefore, the fine particle metal storing the hydrogen can be taken into the cell with maintaining the stored hydrogen, and generate the hydrogen in the cell. The hydrogen is stored, as the atomic hydrogen, by the fine particle of metal or metal alloy. The generated hydrogen may affect on the organism not only as the atomic hydrogen but also as the formed molecular hydrogen. Both of the atomic hydrogen and molecular hydrogen can lead the reduction effect in the organism. Especially, the atomic hydrogen has the stronger reduction power. Thus, it is preferable to make the atomic hydrogen work in the organism.

Magnesium (Mg), titanium (Ti), vanadium (V), lanthanum (La) and the like can be utilized as the metal storing the hydrogen. The hydrogen storing metal or metal alloy in the present invention includes the above metals and their alloyed metals that can store the hydrogen by becoming the hydrides combined with the hydrogen. Among them, the magnesium or its alloyed metal is preferable because it hardly remains in the skin and then it is flowed out. A metal compound storing the hydrogen is called as a hydrogenated metal compound. For example, the magnesium storing the hydrogen is called as a magnesium hydride (MgH₂).

When the magnesium hydride is utilized, it reacts with water coming from the inside of skin, and generates the molecular hydrogen and the remaining of it becomes Mg(OH)₂. Thus, it is safety. As the Mg(OH)₂ is hardly but absolutely soluble in water, it does not remain in the skin. Therefore, it is harmless to the organism because it is flowed out to the organism. The water coming from the inside of skin is moisture internally kept by the skin, moisture generated by the metabolism, sweat or the like.

When the fine particle is the magnesium hydride of atomic hydrogen storing metal or metal alloy, the absorption of atomic hydrogen (H) leads the stronger reduction power by directly passing the magnesium hydride through the superficial skin. Then, the atomic hydrogen generated from MgH₂ reduces a contacted molecule, such as melanin (represented by X in the chemical formula described below, although it is not limited to the melanin).

MgH₂+2X→Mg+2H+2X→Mg+2XH

The remaining Mg reacts with H₂O and becomes Mg(OH)₂. Therefore, it is harmless because it does not remain in the cell as described above.

The fine particle of hydrogen storing metal or metal alloy according to the present invention has the mean particle diameter 0.5 μm-10 μm. Preferably, it has the mean particle diameter 1 μm-5 μm. Further preferably, it has the mean particle diameter 1 μm-3 μm.

Moreover, it is also possible to further micronize (nano-technique) the fine particle and to utilize the nanoparticle having the nanometer size. The nanoparticle has the mean particle diameter 5-500 nm. Preferably, it has the mean particle diameter 10-100 nm. The nanoparticle having the diameter not more than 100 nm can pass the superficial skin of organism and can reach the dermis. In addition, the cell has the property of taking the nanoparticle into it. For example, when the magnesium hydride with nano size is taken into the cell, it emits the hydrogen in the cell and leads the reduction effect. When the magnesium hydride reacts with water, Mg(OH)₂ is formed as the coating. Thus, it reaches the dermis in stable, relatively. Although Mg(OH)₂ is hardly soluble, the whole particle size gradually becomes smaller as it is gradually solved. Hence, if the magnesium hydride is a fine particle having the size that avoids it from reaching the dermis through the skin, the magnesium hydride can be micronized during passing the skin and can reach the dermis. Therefore, even if a fine particle having a size larger than the nano-size is applied to the skin, the fine particle can be gradually micronized, pass the skin and reach the dermis.

When fine particles having different particle diameters are incorporated for different aims, it is possible to work both effects of these different fine particles. When a fine particle having a larger size is incorporated, it generates the hydrogen more slowly. When a fine particle having a smaller size is incorporated, it generates the hydrogen more rapidly due to the larger surface area. Therefore, when both of smaller fine particle and larger fine particle are incorporated, it is possible to generate the hydrogen not only rapidly but also for a long time.

The hydrogen storing metal or metal alloy can be manufactured by binding the hydrogen to the metal. Thus, it can be manufactured by the conventional known technique. For example, a pressure is applied on the condition including the metal and hydrogen, for manufacturing it. The obtained hydrogen storing metal or metal alloy may be micronized by the powder collision or the like.

The specific gravity of MgH₂ is about 1.7. Thus, when MgH₂ having the large particle diameter, e.g., the mean particle diameter not less than tens μm, is suspended in the glycerol having the specific gravity 1.26, the particle is precipitated and it is difficult to keep the homogenous condition. It is not preferable to sell the suspension including the precipitated material as the product, because the user needs stirring when using it. However, when MgH₂ having the mean particle diameter not more than 2 μm is suspended in the glycerol or the oil, such as horse oil, the MgH₂ is not precipitated for a long time and it is possible to keep the homogenous suspension. It is considered that the result is due to the characteristics of fine particle which generates the property of electrically repelling the particles to each other by the charge of electron.

When MgH₂ is suspended in the liquid medium, such as glycerol or oil, to make the homogenous suspension of MgH₂ and the suspension is added to the cosmetics, such as skin lotion, or the water liquid solution, such as drink, the MgH₂ moves down to the lower portion. The molecular hydrogen generated at the lower portion is dissolved in the water liquid solution while moving up to the upper portion. Thus, it is possible to manufacture the hydrogen water with high density, for example, the saturated hydrogen water in which the molecular hydrogens is saturated. On the other hand, if MgH₂ is directly added as the fine particle to the water liquid solution, it reacts and generates the molecular hydrogen at the upper portion of water liquid solution. Thus, the hydrogen is not dissolved into the water liquid solution, adequately. Therefore, it is not possible to manufacture the water dissolving the hydrogen with high density.

Even in the case that the hydrogen storing alloyed metal of magnesium hydride is not the fine particle, the water and MgH₂ particle will react to each other. Then, because the generated Mg(OH)₂ is dissolved in water, the magnesium hydride particle will become smaller and may pass the superficial skin. Different from the metal Mg, the MgH₂ does not react during a short time with the oxygen in the air. Thus, the MgH₂ particle repeatedly collide each other and then are micronized. It is possible to manufacture a particle having the desired size, on the basis of the collision speed and collision frequency. It is also possible to manufacture a fine particle that can pass the superficial skin. For example, it is possible to further micronize the fine particle of MgH₂ by a method, in which the fine particle is put into an airtight container with the inert gas (e.g., nitrogen) as the carrier gas and which causes the collision of particles to each other or the collision to the wall surface. It is possible to obtain the fine particle of MgH₂ having the desired size, by controlling the pressure of carrier gas directed in the circumferential direction, the flow amount thereof, and the added amount of MgH₂ powder, at that time. Among the MgH₂ fine particles having the size not more than the desired particle diameter, the fine particle having the smaller particle diameter moves closer to the center portion by the centrifugal force based on its own apparent specific gravity. Then, it is accompanied by the carrier gas and flowed out of the system. In other words, the energy of motion is applied by the carrier gas to the target substance to be micronized for moving it in the circumferential direction, the particles of target substance are collided with each other, with wall surfaces or with obstacles by the gas flow becoming the eddy flow at any portions, and thus it is possible to further micronize the fine particle of hydrogen storing metal or metal alloy. In addition, it is possible to utilize the wet bead mill for micronizing the magnesium hydride. For example, the particle of magnesium hydride is mixed with oil, the mixture is put into the bead mill, and then the particle of magnesium hydride is made to have the mean particle diameter about 1 μm.

It should be noted that the micronizing method of magnesium hydride described above is an example, and the present invention is not limited to this example.

The fine particle of hydrogen storing metal or metal alloy can be stored as the powder of fine particle, and can be utilized as the composition by mixed with other reagent when needed. In addition, it can be stored by suspended in the anhydrous liquid, such as glycerol or oil, and can be utilized as the composition by mixed with other reagent when needed. It is possible as the oil to select the animal oil, such as horse oil (Bayu), whale oil (Geiyu), lanolin oil (Yoyu) and squalene oil (Sameyu), and the vegetable oil, such as jojoba oil, olive oil, coconut oil and camellia oil (Tsubaki oil). Furthermore, it is possible to select the oil and fat, such as shea butter. In addition, the hydrogen storing metal or metal alloy can be stored while the creamy form or gel type form is kept. It should be noted that the oil described above is not limited to the nature oil, and it is possible to select the artificially synthesized organic compound or the like as the oil.

The metal alloy storing the hydrogen can generate the atomic hydrogen, even without the catalyst. Although the atomic hydrogen itself cannot pass the skin, it can pass the skin as the fine particle form of MgH₂. Then, the fine particle generates the atomic hydrogen in the cell, and the reduction effect is implemented. The generated atomic hydrogen may form the molecular hydrogen, and the molecular hydrogen has the reduction power weaker than the atomic hydrogen, but the formed molecular hydrogen also implements the reduction effect in the organism.

The fine particle of metal alloy storing the hydrogen can be incorporated into the external-use composition. For example, the externally applied composition is the skin external-use composition that can be applied to the skin.

The skin external-use composition has the antioxidant effect, can implement lighting of melanin, and has the whitening effect. Thus, it can be utilized as the whitening composition. Furthermore, it can be utilized as the composition for treatment against the inflammation disease of skin and the allergy disease treatment of skin. It can accelerate the healing of wound, burn injury, bruise or the like, because preventing the inflammation. The inflammation disease described above includes an inflammation disease caused by staphylococcus aureus bacteria, alveolar pyorrhea bacteria or the like, and an inflammation disease caused by wound, scratch injury, burn injury, bruise or the like. The allergy disease described above includes the atopic dermatitis, pollen disease, and the like. In addition, it is possible with the antiallergic effect or the like to prevent itching. Moreover, the skin external-use composition described above has the anti-aging effect.

The present invention includes the skin external-use drug or skin cosmetics having the effects described above, in which the above skin external-use composition is incorporated. Furthermore, the present invention includes the massage supporting liquid, such as massage oil, the esthetic clinic (total beauty technique, such as whitening) supporting liquid, the quasi-drug, the medical product, and the like, in which the above skin external-use composition is incorporated. When the above massage supporting liquid in which the skin external-use composition is incorporated is applied to skin and then the massage is given, the muscle pain and stiff shoulder can be reduced.

It is possible to utilize the common procedure for manufacturing the cosmetics, medical goods, massage supporting liquid, esthetic clinic supporting liquid, quasi-drug, medical product and the like, which include the skin external-use composition of the present invention. Thus, it is possible to manufacture the basic cosmetics, such as skin lotion, milky lotion, moisturizer cream, cleansing cream, massage cream, face wash cream, pack and beauty essence, the bath cosmetics, such as bath agents, the makeup cosmetics, such as foundation, lipstick, mascara and eye shadow, the special purpose cosmetics, such as sunscreen, the after-shave lotion, the body soap, and the like. It is preferable to make these products in the cream form or gel form. In addition, other various components may be incorporated. For example, it is possible for the cosmetics to incorporate oil, surfactants, humectants, thickener, preservatives, antioxidants, ultraviolet absorbers, ultraviolet scattering agent, flavors, dyes, pigments, bees wax or the like. As the oil, it is possible to utilize the animal oil, such as horse oil and whale oil, the vegetable oil, such as jojoba oil and coconut oil, the fish oil, the bird oil and the like. The metal or metal alloy storing the hydrogen of the present invention takes time to generate the hydrogen in the condition of strong alkali whose pH is not less than 11. Thus, it is preferable to select the pH of weak alkali, neutral or acidic, for the above composition or the cosmetics, medical goods, massage supporting liquid, esthetic clinic supporting liquid and quasi-drug which include the above composition. Particularly, the pH should be less than 11. Preferably, the pH should be less than and close to 10. However, it is not necessary to consider the pH change after the generation of hydrogen. For example, it is not required to prepare the solid acid or the like to neutralize.

When the magnesium hydride is suspended and applied with homogenous smooth liquid solution or creamy material directly onto the skin, the hydrogen is absorbed in the skin. The magnesium hydride reacts with water included in the skin or generated from the skin cell, to generate the hydrogen, and the generated hydrogen is absorbed in the organisms through the skin before dispersing into the air. After reacting with water, the magnesium hydride becomes the Mg(OH)₂. Although hardly, the Mg(OH)₂ is water soluble. Thus, it is easily absorbed or flowed out. Therefore, it is safety because no disorder is caused on the skin.

When the fine particle is utilized, it is possible to obtain the stronger whitening effect and stronger antioxidant effect. A particle becomes smaller, as reacting with water, and then becomes the fine particle.

For example, in the case that the composition of the present invention is utilized for the massage supporting liquid or esthetic clinic supporting liquid, the 1-5% (wt/vol) of MgH₂ is suspended into the oil, such as horse oil, in order to apply it on the skin at the time when the massage or esthetic clinic is started. The amount of MgH₂ may be changed adequately in consideration of the personal tolerance or the duration of the effect.

In addition, it is possible to manufacture the cream entrapping the MgH₂ by suspending the MgH₂ into the heated mixture of oil, such as jojoba oil, and bee wax, and then by cooling the suspension. The adjustment of amount of bee wax can change the firmness of cream. In addition, the flavor can be added. It is further possible to utilize not only the oil and bee wax but also emulsifying wax, shea butter or the like.

For example, the medical products incorporated with the skin external-use composition of the present invention is a cosmetic face mask (face sheet, foundation, face pack sheet or the like), bandage or libatape (adhesive tape) which include magnesium storing the hydrogen of the present invention. For example, the hydrogen storing magnesium is applied to the fabric portion of the face mask or bandage. When the face mask or bandage is worn, the hydrogen storing magnesium contacts with the skin, reacts with water of skin, and generates the hydrogen. Then, the hydrogen is taken into the skin cell. Water may be externally applied by the aerosol or the like, at that time.

The MgH₂ can generate the molecular hydrogen under the room temperature or body temperature, even without the catalyst. Therefore, the catalyst is not necessary for generating the hydrogen by the fine particle of hydrogen storing metal or metal alloy according to the present invention.

When added dropwise to the water liquid solution, such as skin lotion, in order to mix, the homogenous suspension with the fine particle of magnesium hydride in the anhydrous liquid, such as glycerol or oil can become the liquid dissolving the molecular hydrogen with high density under the room temperature even without the catalyst. Furthermore, when added to the drink, such as coffee, tea, juice, miso soup, soup, milk, or liquor, the anhydrous liquid containing the fine particle of magnesium hydride generates the molecular hydrogen in the drink. Therefore, it is possible to obtain the drink dissolving the molecular hydrogen with high density. Furthermore, it is possible to intake the molecular hydrogen together with liquid, for the sake of the drink. Only by adding the magnesium hydride to the drink and keeping it under the room temperature for 1 minute at that time, it is possible to manufacture the drink in which the hydrogen is saturated.

The skin lotion containing saturated hydrogen can be utilized for cosmetics, such as skin beautification. The drink containing saturated hydrogen also can be utilized as healthy food, functional food, special health food, food supplement and the like which have antioxidant effect.

For example, when the magnesium hydride particle having the particle diameter 2 μm is suspended in the dehydrated glycerol to make the MgH₂ become 3.3% (wt/vol), it is possible to manufacture the saturated hydrogen water liquid solution by adding one drop of this suspension (about 50 μL) into 100 mL of skin lotion or drink. At that time, not less than 50% of generated hydrogen is dissolved in water. It does not cause the problem that the extra hydrogen gas brings the burning or explosion.

### Embodiment

Although the present invention is particularly explained with the embodiments described below, the present invention is not limited to the embodiments.

### Embodiment 1

### Manufacturing the Fine Particle of MgH₂

The particle of MgH₂ having the mean particle diameter 60 μm is micronized to make the mean particle diameter become 2 μm, and thus the fine particle of MgH₂ is manufactured.

Furthermore, the fine particle of MgH₂ having the mean particle diameter 2 μm is micronized to make the mean particle diameter become 10-100 nm, and thus the nanoparticle of MgH₂ is manufactured. In the following embodiments, the term of particle diameter represents the mean particle diameter.

In addition, the particle of MgH₂ having the mean particle diameter 60 μm is micronized by the wet beads mill, and thus the fine particle of MgH₂ having the mean particle diameter 1.035 μm is manufactured.

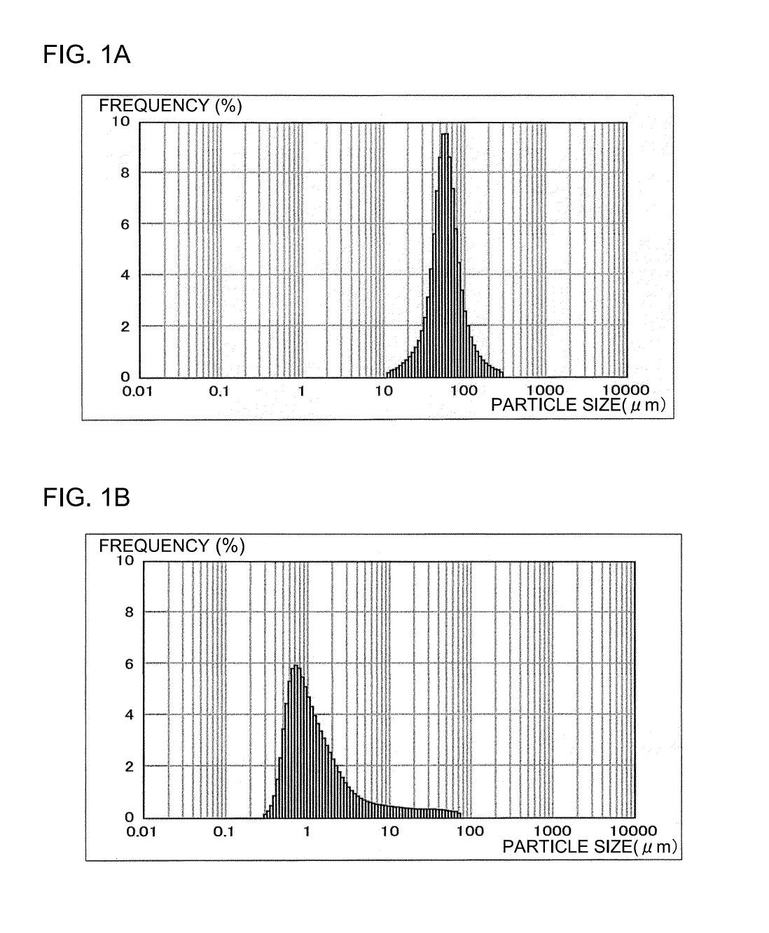

FIG. 1A is a graph that shows a distribution of particle diameter of MgH₂ particle before micronized. FIG. 1B is a graph that shows a distribution of particle diameter of MgH₂ particle after micronized. The horizontal axis represents the particle diameter of MgH₂ particle, and the vertical axis represents the frequency. For example, when 1 kg of MgH₂ particle having the mean particle diameter 60 μm is mixed with 4 kg of jojoba oil, put into the wet beads mill and processed with 1.5-2.5 l/m flow rate as shown in FIG. 1A, it is possible to obtain the MgH₂ particle having the mean particle diameter 1.035 μm as shown in FIG. 1B.

It should be noted that the utilized liquid is an example. Thus, it is also possible to utilize the olive oil, horse oil or the like. When the olive oil is utilized, it is possible to obtain the MgH₂ particle having the mean particle diameter 1.34 μm. Hence, it is possible to obtain the MgH₂ particle having the size smaller by the wet beads mill than the dry method described above.

### Embodiment 2

### The Lighting Effect for Melanin

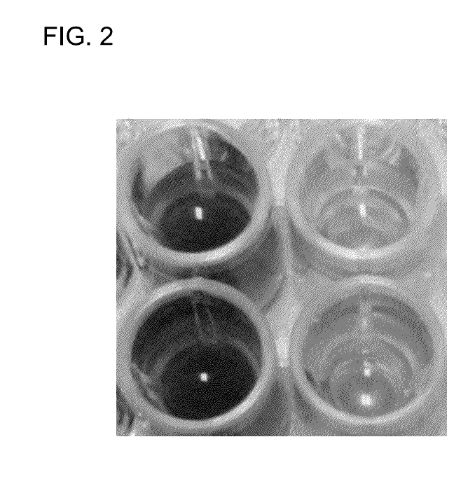

After the melanin (Sigma-Aldrich) is dissolved in the weak alkali liquid solution, the solution is neutralized by the dilute acetic acid and then the glycerol suspension containing 10% of MgH₂ having the particle diameter 2 μm is dropped to the neutralized solution. In addition, the glycerol suspension containing 10% of MgH₂ having the particle diameter 2 μm is previously reacted with water, and then the reacted suspension from which the hydrogen is emitted is added dropwise to the above melanin liquid solution, in order to prepare the control sample. Some of them are put on the microplate and the images are captured. FIG. 2 shows the result. In FIG. 2, the image at the left-top shows the control sample in which the MgH₂ previously reacted with water is added to the melanin liquid solution. The image at the right-top shows the melanin liquid solution treated with MgH₂. The image at the left-lower shows the melanin liquid solution treated with the hydrogen gas. The image at the right-lower shows the melanin liquid solution treated with MgH₂. As shown in FIG. 2, the lighting of melanin is observed in the MgH₂ treatment. On the other hand, the color change is not observed in the control sample.

### Embodiment 3

### The Whitening Effect

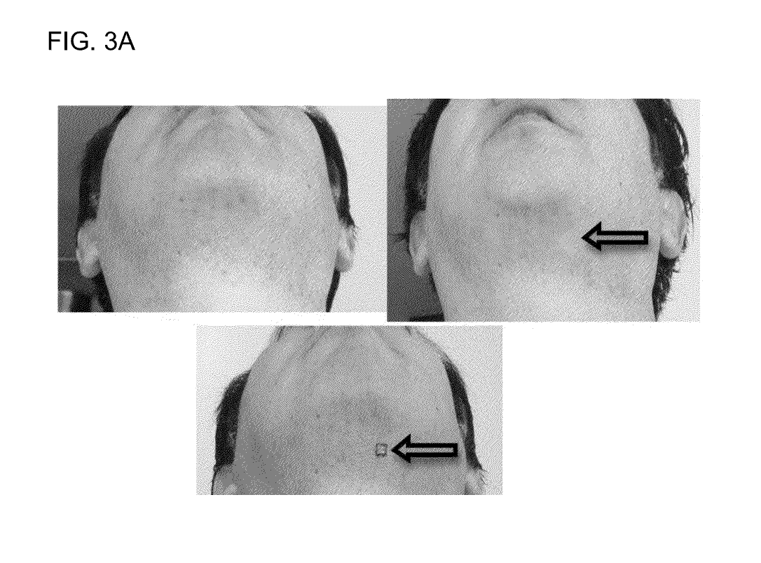

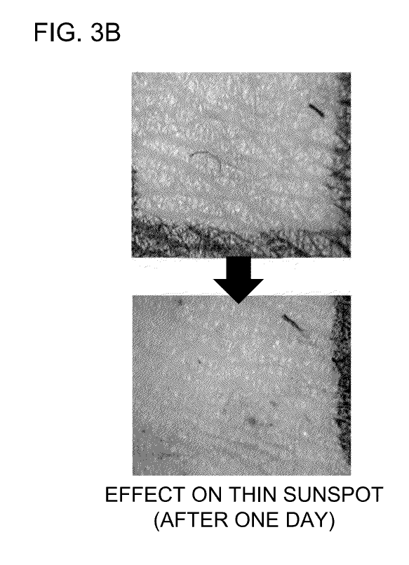

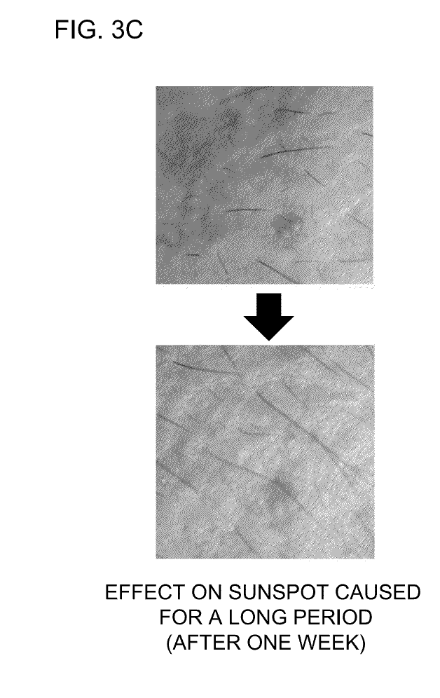

Cream containing 10% (wt/vol) of MgH₂ having the particle diameter 2 μm is applied to the skin, and then the image is captured after 24 hours. The left image in FIG. 3A shows the skin condition before the application of cream. The right image in FIG. 3A shows the skin condition after the application of cream. The lower image in FIG. 3A shows a position where the cream is applied. The arrow represents the applied position of cream. The position becomes whiter than the area around the position.

FIG. 3B shows another experimental result, in which cream containing 10% (wt/vol) of MgH₂ having the particle diameter 2 μm is applied to the skin and then the image is captured after 24 hours. The top image in FIG. 3B shows the skin condition before the application of cream. The lower image in FIG. 3B shows the skin condition after the application of cream. As shown in each image of FIG. 3B, it is observed that the sunspot at the almost center portion is disappeared and whitened.

FIG. 3C shows another experimental result, in which cream containing 10% (wt/vol) of MgH₂ having the particle diameter 2 μm is applied to the skin once a day and then the image is captured after 1 week. The top image in FIG. 3C shows the skin condition before the application of cream. The lower image in FIG. 3C shows the skin condition after the application of cream. As shown in each image of FIG. 3C, it is observed that the sunspot at the left-upper portion is disappeared and whitened.

### Embodiment 4

### The Effect for Wound

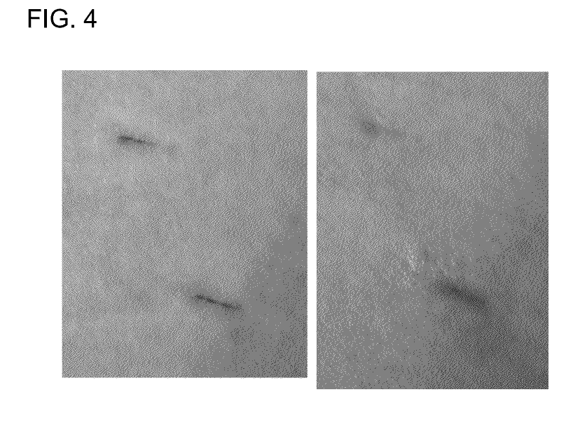

Some injuries caused by the scratch of cat become similar scars without moving the positions. The left image in FIG. 4 is immediately after the cat scratches, and degrees of two injuries are similar to each other. Cream containing 1.4% (wt/vol) of MgH₂ having the particle diameter 2 μm is applied only to the upper portion. On the other hand, cream containing 10% (wt/vol: the number of moles is adjusted for MgH₂) of Mg(OH)₂, as the control sample, is applied to the lower portion. One day after the application, both portions are compared (right in FIG. 4). Not only the difference of appearances shown in images but also other difference was observed. The injury at the upper portion did not cause the inflammation that is common for the cut injury and brings something like fever. In addition, the scar at the upper portion after the healing was disappeared earlier. Therefore, the differences were obvious.

### Embodiment 5

### The Effect for Burn Injury

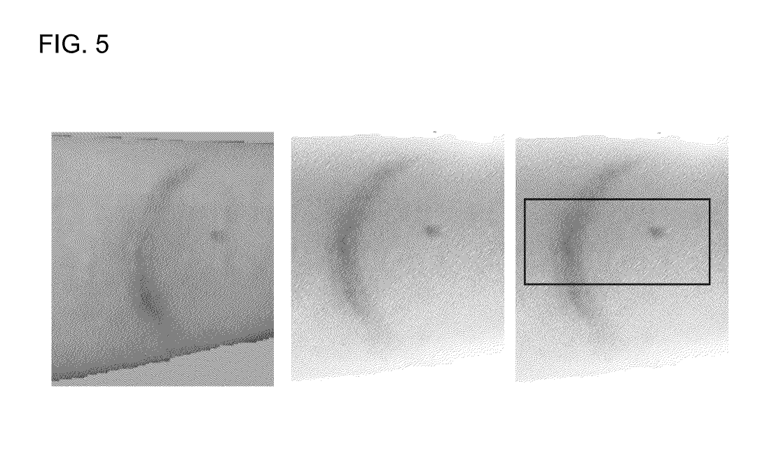

Cream containing 1.4% (wt/vol) of MgH₂ having the particle diameter 2 μm is applied only to the center portion of blackened scar area due to the burn injury. Before the application (the center image in FIG. 5), the center portion is blackened at the most. However, it is observed that only the center portion is lightened after 1 day (the left image in FIG. 5). The right image in FIG. 5 shows the applied area of MgH₂ cream.

### Embodiment 6

### The Effect for Bruise

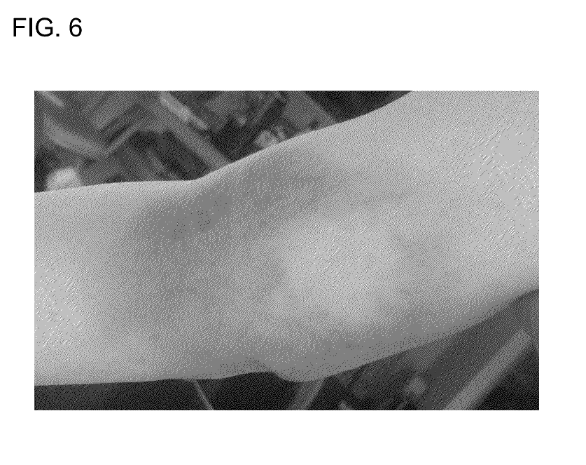

The subject person bumped his upper arm to the door of car. After several hours, cream containing 1.4% (wt/vol) of MgH₂ having the particle diameter 2 μm was applied to a portion of bumped area, 1 cm² of skin, and then the bumped area is covered by an adhesive tape. After 1 day, a photograph of the bruise area was taken. Only the area applied with MgH₂ was not blackened. Thus, the anti-inflammatory effect is shown (FIG. 6).

### Embodiment 7

### The Effect for Itching Due to the Mosquito Bite

(1) After bitten by a mosquito, the subject person sit for 5-8 minutes in the bath filled with water in which MgH₂ having the particle diameter 60 μm is dissolved to generate the hydrogen. Then, the subject person felt free from the itching (0.8-1 g of MgH₂ was applied into the bath filled with 200 litter of water [40-41 C.°], and 5 minutes later, the subject person sit in the bath).
(2) After bitten by a mosquito, MgH₂ having the particle diameter 2 μm was applied to the bitten area. Then, the subject person felt free from the itching.

These results indicate that the hydrogen storing metal has the effect for treating the allergy.

### Embodiment 8

### The Treating for Athlete's Foot (Tinea Pedis)

A patient of athlete's foot is treated for ten months with the bath (prepared with the same method to the embodiment 7) in which MgH₂ having the particle diameter 60 μm is dissolved to generate the hydrogen (the patient took the bath for 10-20 minutes, with 5-7 times once a week). Then, the athlete's foot was healed.

### Embodiment 9

### The Measurement of Hydrogen Density

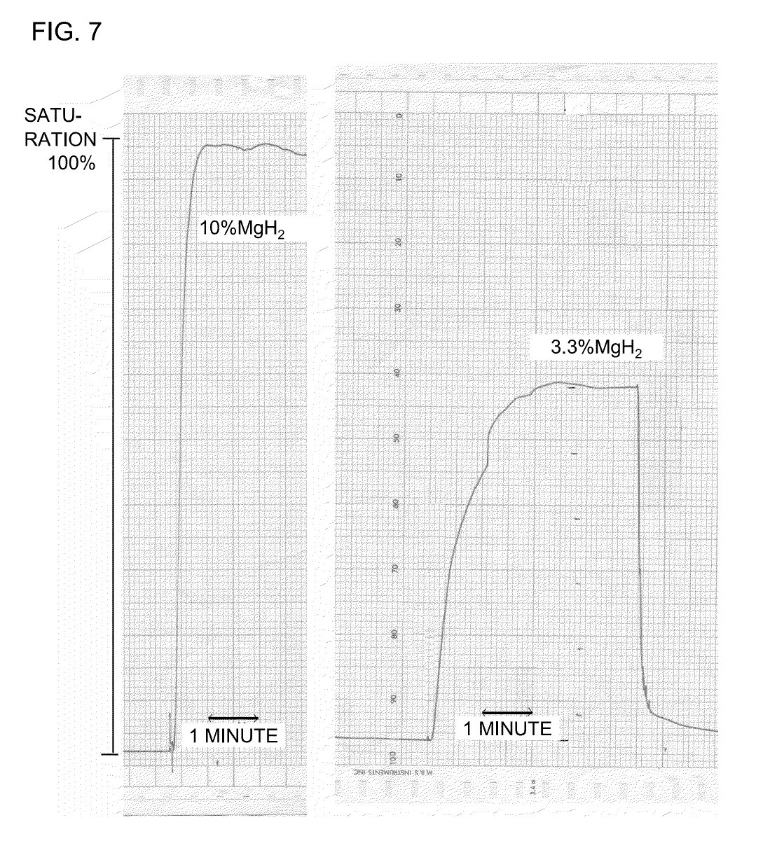

After glycerol suspension containing the MgH₂ having the particle diameter 2 μm was added dropwise to 150 mL of water (pH 6.8), the added water was stirred soon. The hydrogen density was measured with the hydrogen electrode, and the temporal response was recorded by the recorder. The speed 2 cm/minute was utilized for the recorder. FIG. 7 shows the chart. In the case that one drop of 10% MgH₂ was added, 100% saturation was observed in the liquid solution after 30 seconds (the left chart in FIG. 7). In the case that one drop of 3.3% MgH₂ was added, 60% saturation was observed in the liquid solution after 2 minutes (the right chart in FIG. 7).

### INDUSTRIAL APPLICABILITY

It is possible to utilize the skin external-use composition that is incorporated with the hydrogen storing metal alloy and generates the hydrogen of the present invention, and utilize the cosmetics, quasi-drug, medical product and the like that are incorporated with the skin external-use composition, for the following purposes:

(1) they can be utilized as the cosmetics having the antioxidant effect, anti-inflammatory effect and antiallergic effect;
(2) they can be utilized as the cream having anti-inflammation effect;
(3) they can improve the inflammation portion;
(4) they can show the antiallergic property based on the antioxidant effect;
(5) they can be utilized as the massage oil for reducing or preventing the muscle pain;
(6) they can be utilized as the cosmetics and the esthetic clinic's liquid showing the anti-aging effect based on the antioxidant effect;
(7) they can improve the blood flow and ameliorate the disease (stiff shoulder, oversensitive to cold);
(8) they can show the analgesic effect(the pain is disappeared when they are utilized as lip cream);
(9) they can be utilized as the composition incorporated into the facial washing liquid or facial washing powder (the composition is solved in water to be the facial washing liquid). When the face is washed with the liquid, the skin's elasticity (anti-saccharification effect) is improved;
(10) they can be utilized for treating the severe alveolar pyorrhea and stomatitis;
(11) they can be utilized for treating the acne/pimple; and
(12) they can reduce the fatigue when applied to the eyelids (improvement effect for the dark circle).

As this invention may be embodied in several forms without departing from the spirit of essential characteristics thereof, the present embodiments are therefore illustrative and not restrictive, since the scope of the invention is defined by the appended claims rather than by the description preceding them, and all changes that fall within metes and bounds of the claims, or equivalence of such metes and bounds thereof are therefore intended to be embraced by the claims.

1-13. (canceled)

14. A skin external-use composition incorporated with a fine particle of metal or metal alloy storing hydrogen.

15. The skin external-use composition according to claim 14, wherein
the fine particle of metal or metal alloy storing hydrogen is a fine particle of magnesium hydride storing atomic hydrogen.

16. The skin external-use composition according to claim 15, which is incorporated with a fine particle of magnesium hydride that can generate molecular hydrogen without catalyst when applied to a skin.

17. The skin external-use composition according to claim 15, which is incorporated with a fine particle of magnesium hydride storing atomic hydrogen that passes a superficial skin.

18. The skin external-use composition according to claims 14, which is in a cream state or gel state.

19. The skin external-use composition according to claims 14, which is utilized for whitening.

20. The skin external-use composition according to claims 14, which is utilized for treating a disorder selected from a group of inflammation, allergy, wound, burn injury and bruise.

21. The skin external-use composition according to claims 14, which is selected from a group of cosmetics, massage supporting liquid, esthetic clinic supporting liquid, quasi-drug and medical product.

22. A homogenous composition that includes a fine particle of magnesium hydride, wherein
the fine particle of magnesium hydride is homogenously suspended in an anhydrous liquid.

23. The composition homogenously suspended in the anhydrous liquid according to claim 22 for manufacturing a liquid solution having a high density of dissolved molecular hydrogen.

24. The composition according to claim 22, wherein
the anhydrous liquid is glycerol or oil.

25. A method for manufacturing a water liquid solution including hydrogen, comprising applying the composition according to claims 22 into a water liquid solution

26. The method according to claim 25, wherein
the water liquid solution is a drink or cosmetics.
